(19) United States
(12) Patent Application Publication
(10) Pub. No.: US 2013/0338061 A1
(43) Pub. Date: Dec. 19, 2013

(54) Salts and Polymorphs of Sulfamide NS3 Inhibitors

(71) Applicants: Lipa Shah, Waltham, MA (US); Paul Allen Sutton, Parsippany, NJ (US); Lijun Zhang, Shanghai (CN)

(72) Inventors: Lipa Shah, Waltham, MA (US); Paul Allen Sutton, Parsippany, NJ (US); Lijun Zhang, Shanghai (CN)

(21) Appl. No.: 13/877,855

(22) PCT Filed: Oct. 8, 2011
(86) PCT No.: PCT/CN2011/080534

§ 371 Date: Sep. 6, 2013

Related U.S. Application Data
(60) Provisional application No. 61/391,456, filed on Oct. 8, 2010.

(51) Int. Cl. C07K 7/06

(52) U.S. Cl. CPC C07K 7/06
USPC 514/4.3; 530/330

(57) ABSTRACT

The invention provides new salts and polymorphs of (5R,8S)-7-[(2S)-2-{[(2S)-2-cyclohexyl-2-({[(2S)-1-isopropylpiperidin-2-yl]carbonyl}amino)acetyl]amino}-3,3-dimethyl butanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide (referred to herein as Compound X), pharmaceutical compositions containing them and processes for their manufacture and use in therapy.

The present invention relates to salts and polymorphs of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide, pharmaceutical compositions containing them and their use in therapy.

### BACKGROUND

Chronic hepatitis C virus (HCV) infection is a major global health burden, with an estimated 170 million people infected worldwide and an additional 3 to 4 million infected each year (See e.g. World Health Organization Fact Sheet No. 164. October 2000). Although 25% of new infections are symptomatic, 60-80% of patients will develop chronic liver disease, of whom an estimated 20% will progress to cirrhosis with a 1-4% annual risk of developing hepatocellular carcinoma (See e.g. World Health Organization Guide on Hepatitis C. 2002; Pawlotsky, J-M. (2006) Therapy of Hepatitis C: From Empiricism to Eradication. Hepatology 43:S207-S220). Overall, HCV is responsible for 50-76% of all liver cancer cases and two thirds of all liver transplants in the developed world (See e.g. World Health Organization Guide on Viral Cancers. 2006). And ultimately, 5-7% of infected patients will die from the consequences of HCV infection (See e.g. World Health Organization Guide on Hepatitis C. 2002).

The current standard therapy for HCV infection is pegylated interferon alpha (IFN-α) in combination with ribavirin. However, only up to 50% of patients with genotype 1 virus can be successfully treated with this interferon-based therapy. Moreover, both interferon and ribavirin can induce significant adverse effects, ranging from flu-like symptoms (fever and fatigue), hematologic complications (leukopenia, thrombocytopenia), neuropsychiatric issues (depression, insomnia, irritability), weight loss, and autoimmune dysfunctions (hypothyroidism, diabetes) from treatment with interferon to significant hemolytic anemia from treatment with ribavirin. Therefore, more effective and better tolerated drugs are still greatly needed.

NS3, an approximately 70 kDa protein, has two distinct domains: a N-terminal serine protease domain of 180 amino acids (AA) and a C-terminal helicase/NTPase domain (AA 181 to 631). The NS3 protease is considered a member of the chymotrypsin family because of similarities in protein sequence, overall three-dimensional structure and mechanism of catalysis. The HCV NS3 serine protease is responsible for proteolytic cleavage of the polyprotein at the NS3/NS4A, NS4A/NS4B, NS4B/NS5A and NS5A/NS5B junctions (See e.g. Bartenschlager, R., L. et al. (1993) J. Virol. 67:3835-3844; Grakoui, A. et al. (1993) J. Virol. 67:2832-2843; Tomei, L. et al. (1993) J. Virol. 67:4017-4026). NS4A, an approximately 6 kDa protein of 54 AA, is a co-factor for the serine protease activity of NS3 (See e.g. Failla, C. et al. (1994) J. Virol. 68:3753-3760; Tanji, Y. et al. (1995) J. Virol. 69:1575-1581). Autocleavage of the NS3/NS4A junction by the NS3/NS4A serine protease occurs intramolecularly (i.e., cis) while the other cleavage sites are processed intermolecularly (i.e., trans). It has been demonstrated that HCV NS3 protease is essential for viral replication and thus represents an attractive target for antiviral chemotherapy.

There remains a need for new treatments and therapies for HCV infection, as well as HCV-associated disorders. There is also a need for compounds useful in the treatment or prevention or amelioration of one or more symptoms of HCV, as well as a need for methods of treatment or prevention or amelioration of one or more symptoms of HCV. Furthermore, there is a need for new compounds capable of modulating the activity of HCV-serine proteases, particularly the HCV NS3/NS4a serine protease and using said compounds to treat, prevent or ameliorate HCV infection.

Unpublished patent application no. PCT/IB2010/000784 describes a novel class of compounds useful in the treatment of HCV-associated disorders. One such compound is (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide, having the structure shown below. This compound is referred to herein as Compound X.

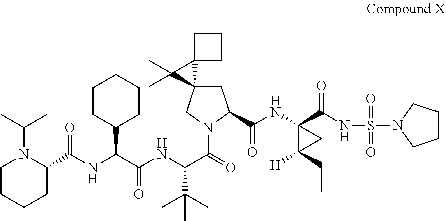 [SMILES: CC[C@@H]1C[C@]1(NC(=O)[C@@H]1C[C@@]2(CN1C(=O)[C@@H](NC(=O)[C@@H](NC(=O)[C@@H]1CCCCN1C(C)C)C1CCCCC1)C(C)(C)C)C(C)(C)C21CCC1)C(=O)NS(=O)(=O)N1CCCC1]

In the manufacture of pharmaceutical formulations, it is important that the active compound be in a form in which it can be conveniently handled and processed in order to obtain a commercially viable manufacturing process. Accordingly, the chemical stability and the physical stability of the active compound are important factors. The active compound, and formulations containing it, must be capable of being effectively stored over appreciable periods of time, without exhibiting any significant change in the physico-chemical characteristics (e.g. chemical composition, density, hygroscopicity and solubility) of the active compound.

Furthermore, if the active compound is to be incorporated into a dosage form for oral administration, such as a tablet, it is desirable that the active compound be readily micronised to yield a powder with good flow properties to aid manufacture.

It is generally found that there are advantages in manufacturing a particular solid-state form of a pharmaceutical ingredient and these are described in “Handbook of Pharmaceutical Salts; Properties, Selection and Use”, P. Heinrich Stahl, Camille G. Wermuth (Eds.) (Verlag Helvetica Chimica Acta, Zurich). Methods of manufacturing solid-state forms are also described in “Practical Process Research and Development”, Neal G. Anderson (Academic Press, San Diego) and “Polymorphism: In the Pharmaceutical Industry”, Rolf Hilfiker (Ed) (Wiley VCH).

The present inventors have discovered a number of salts and crystalline polymorphs of Compound X. Salt and crystal formation has the potential to improve yields as the resultant new physical form may exhibit lower solubility in organic solvents. This can lead to an improved manufacturing process. Additionally, the salts may be easier to isolate and purify. Thus, in one aspect, the invention provides Compound X hydrochloride salt, or a pharmaceutically acceptable derivative thereof. In an embodiment, the purity of the salt is at least 98%. In another embodiment, the purity of the salt is at least 99%. In another aspect, the invention provides Compound X hemi-hydrochloride salt, or a pharmaceutically acceptable derivative thereof. In another aspect, the invention provides Compound X methanesulfonic acid salt, or a pharmaceutically acceptable derivative thereof. In yet another aspect, the invention provides Compound X succinic acid salt, or a pharmaceutically acceptable derivative thereof.

Furthermore, in accordance with the present invention, there are provided a number of crystalline polymorphs of Compound X and its salts.

Thus, in one aspect, the invention provides a crystalline form of Compound X hydrochloride salt (Form A) which exhibits at least the following characteristic X-ray powder diffraction peaks (expressed in degrees 2θ): 7.7, 8.9, 11.8, 15.5 and 18.0. In one embodiment, Form A exhibits at least the following characteristic X-ray powder diffraction peaks: 7.7, 8.9, 11.8, 15.5, 17.3, 18.0 and 19.9. In another embodiment, Form A exhibits at least the characteristic X-ray powder diffraction peaks shown in Table A. In yet another embodiment, Form A exhibits an X-ray powder diffraction pattern substantially the same as that shown in FIG. 1.

In another aspect, the invention provides a crystalline form of Compound X hydrochloride salt (Form B) which exhibits at least the following characteristic X-ray powder diffraction peaks (expressed in degrees 2θ): 7.8, 8.6, 11.3, 15.8 and 18.2. In one embodiment, Form B exhibits at least the following characteristic X-ray powder diffraction peaks: 7.8, 8.6, 9.5, 11.3, 14.8, 15.8 and 18.2. In another embodiment, Form B exhibits at least the characteristic X-ray powder diffraction peaks shown in Table B. In yet another embodiment, Form B exhibits an X-ray powder diffraction pattern substantially the same as that shown in FIG. 2.

In another aspect, the invention provides a crystalline form of Compound X hydrochloride salt (Form C) which exhibits at least the following characteristic X-ray powder diffraction peaks (expressed in degrees 2θ): 9.2, 14.4, 15.3, 17.7 and 20.0. In one embodiment, Form C exhibits at least the following characteristic X-ray powder diffraction peaks: 8.0, 9.2, 14.4, 15.3, 17.7, 19.3 and 20.0. In another embodiment, Form C exhibits at least the characteristic X-ray powder diffraction peaks shown in Table C. In yet another embodiment, Form C exhibits an X-ray powder diffraction pattern substantially the same as that shown in FIG. 3.

In another aspect, the invention provides a crystalline form of Compound X hydrochloride salt (Form D) which exhibits at least the following characteristic X-ray powder diffraction peaks (expressed in degrees 2θ): 8.0, 8.3, 14.3, 15.9 and 18.2. In one embodiment, Form D exhibits at least the following characteristic X-ray powder diffraction peaks: 8.0, 8.3, 9.1, 10.0, 10.7, 14.3, 14.8, 15.9, 17.2 and 18.2. In another embodiment, Form D exhibits at least the characteristic X-ray powder diffraction peaks shown in Table D. In yet another embodiment, Form D exhibits an X-ray powder diffraction pattern substantially the same as that shown in FIG. 4.

In another aspect, the invention provides a crystalline form of Compound X hydrochloride salt (Form E) which exhibits at least the following characteristic X-ray powder diffraction peaks (expressed in degrees 2θ): 7.5, 8.8, 9.2, 15.6 and 17.8. In one embodiment, Form E exhibits at least the following characteristic X-ray powder diffraction peaks: 7.5, 8.8, 9.2, 15.6, 17.8, 18.2 and 19.5. In another embodiment, Form E exhibits at least the characteristic X-ray powder diffraction peaks shown in Table E. In yet another embodiment, Form E exhibits an X-ray powder diffraction pattern substantially the same as that shown in FIG. 5.

In another aspect, the invention provides a crystalline form of Compound X hemi-hydrochloride salt (Form F) which exhibits at least the following characteristic X-ray powder diffraction peaks (expressed in degrees 2θ): 6.9, 11.3, 14.8, 16.0 and 18.2. In one embodiment, Form F exhibits at least the following characteristic X-ray powder diffraction peaks: 6.9, 7.8, 9.1, 11.3, 14.8, 16.0, 17.4 and 18.2. In another embodiment, Form F exhibits at least the characteristic X-ray powder diffraction peaks shown in Table F. In yet another embodiment, Form F exhibits an X-ray powder diffraction pattern substantially the same as that shown in FIG. 6.

In another aspect, the invention provides a crystalline form of Compound X methanesulfonic acid salt (Form G) which exhibits at least the following characteristic X-ray powder diffraction peaks (expressed in degrees 2θ): 5.8, 6.5, 7.8, 10.4 and 15.7. In one embodiment, Form G exhibits at least the following characteristic X-ray powder diffraction peaks: 5.8, 6.5, 7.8, 10.4, 12.9, 15.7 and 17.2. In another embodiment, Form G exhibits at least the characteristic X-ray powder diffraction peaks shown in Table G. In yet another embodiment, Form G exhibits an X-ray powder diffraction pattern substantially the same as that shown in FIG. 7.

In another aspect, the invention provides a crystalline form of Compound X (Form H) which exhibits at least the following characteristic X-ray powder diffraction peaks (expressed in degrees 2θ): 5.7, 6.9, 7.8, 14.4 and 18.5. In one embodiment, Form H exhibits at least the following characteristic X-ray powder diffraction peaks: 5.7, 6.9, 7.8, 9.2, 10.3, 12.4, 14.4, 15.5, 16.3, 16.7 and 18.5. In another embodiment, Form H exhibits at least the characteristic X-ray powder diffraction peaks shown in Table H. In yet another embodiment, Form H exhibits an X-ray powder diffraction pattern substantially the same as that shown in FIG. 8.

In another aspect, the invention provides a crystalline form of Compound X (Form I) which exhibits at least the following characteristic X-ray powder diffraction peaks (expressed in degrees 2θ): 9.5, 13.3, 14.5, 19.0 and 19.7. In one embodiment, Form I exhibits at least the following characteristic X-ray powder diffraction peaks: 6.8, 7.4, 7.9, 9.5, 13.3, 14.0, 14.5, 16.1, 17.9, 19.0 and 19.7. In another embodiment, Form I exhibits at least the characteristic X-ray powder diffraction peaks shown in Table I. In yet another embodiment, Form I exhibits an X-ray powder diffraction pattern substantially the same as that shown in FIG. 9.

In another aspect, the invention provides a crystalline form of Compound X (Form J) which exhibits at least the following characteristic X-ray powder diffraction peaks (expressed in degrees 2θ): 6.9, 8.3, 12.5, 13.6, 16.0, 16.8, and 17.1. In one embodiment, Form J exhibits at least the following characteristic X-ray powder diffraction peaks: 6.9, 8.3, 9.2, 12.5, 13.6, 16.0, 16.8, 17.1, 19.8 and 20.9. In another embodiment, Form J exhibits at least the characteristic X-ray powder diffraction peaks shown in Table J. In yet another embodiment, Form J exhibits an X-ray powder diffraction pattern substantially the same as that shown in FIG. 10.

In another aspect, the invention provides a crystalline form of Compound X (Form K) which exhibits at least the following characteristic X-ray powder diffraction peaks (expressed in degrees 2θ): 6.1, 7.4, 8.3, 22.1, 23.7, 24.1, and 24.6. In one embodiment, Form K exhibits at least the following characteristic X-ray powder diffraction peaks: 5.2, 6.1, 7.4, 8.3, 9.7, 18.2, 19.6, 22.1, 23.2, 23.7, 24.1, and 24.6. In another embodiment, Form K exhibits at least the characteristic X-ray powder diffraction peaks shown in Table K. In yet another embodiment, Form K exhibits an X-ray powder diffraction pattern substantially the same as that shown in FIG. 11.

In another aspect, the invention provides a crystalline form of Compound X (Form L) which exhibits at least the following characteristic X-ray powder diffraction peaks (expressed in degrees 2θ): 5.7, 8.2, 16.9, 18.4 and 18.5. In one embodiment, Form L exhibits at least the following characteristic X-ray powder diffraction peaks: 5.7, 7.0, 8.2, 15.4, 16.0, 16.9, 18.4 and 18.5. In another embodiment, Form L exhibits at least the characteristic X-ray powder diffraction peaks shown in Table L. In yet another embodiment, Form L exhibits an X-ray powder diffraction pattern substantially the same as that shown in FIG. 12.

In another aspect, the invention provides a crystalline form of Compound X (Form M) which exhibits at least the following characteristic X-ray powder diffraction peaks (expressed in degrees 2θ): 6.7, 7.6, 7.7, 9.5, and 19.0. In one embodiment, Form M exhibits at least the following characteristic X-ray powder diffraction peaks: 6.7, 7.6, 7.7, 9.5, 13.1, 14.4, 15.4, 16.0, 17.8, 18.3, 19.0 and 19.6. In another embodiment, Form M exhibits at least the characteristic X-ray powder diffraction peaks shown in Table M. In yet another embodiment, Form M exhibits an X-ray powder diffraction pattern substantially the same as that shown in FIG. 13.

In another aspect, the invention provides a crystalline form of Compound X (Form N) which exhibits at least the following characteristic X-ray powder diffraction peaks (expressed in degrees 2θ): 6.3, 7.7, 8.7, 16.0, 18.1, and 20.5. In one embodiment, Form N exhibits at least the following characteristic X-ray powder diffraction peaks: 6.3, 7.7, 8.7, 10.2, 11.4, 13.8, 16.0, 17.2, 18.1, 18.6, 19.0 and 20.5. In another embodiment, Form N exhibits at least the characteristic X-ray powder diffraction peaks shown in Table N. In yet another embodiment, Form N exhibits an X-ray powder diffraction pattern substantially the same as that shown in FIG. 14.

In one aspect of the invention, the polymorphs of the invention have crystalline properties and are preferably at least 50% crystalline, more preferably at least 60% crystalline, still more preferably at least 70% crystalline and most preferably at least 80% crystalline. Crystallinity can be estimated by conventional X-ray diffractometry techniques or by infra-red spectroscopic techniques.

In one aspect of the invention, the polymorphs of the invention are from 50%, 60%, 70%, 80% or 90% to 95%, 96%, 97%, 98%, 99% or 100% crystalline.

In the present specification, X-ray powder diffraction peaks (expressed in degrees 2θ) are measured using copper X-rays with a wavelength of 1.5406 Å (alpha1) and 1.5444 Å (alpha2).

The crystalline forms of the present invention can exist in both unsolvated and solvated forms. The term ‘solvate’ is used herein to describe a molecular complex comprising the compound of the invention and an amount of one or more pharmaceutically acceptable solvents. Examples of pharmaceutically acceptable solvents include ethanol and water. The term ‘hydrate’ is employed when the solvent is water.

In one aspect, the invention provides a salt or crystalline form defined herein for use in therapy. In another aspect, the invention provides a method of treatment by therapy, comprising administering to a subject in need thereof a pharmaceutically acceptable amount of a salt or crystalline form of the invention.

In one aspect, the invention provides the use of a salt or crystalline form defined herein in the manufacture of a medicament for use in therapy.

In one embodiment, the therapy is the treatment of an HCV-associated disorder. In another embodiment, the therapy is the treatment of an HIV infection. In another embodiment the therapy is the treatment, inhibition or prevention of the activity of HCV. In another embodiment, the therapy is the inhibition of the activity of the NS2 protease, the NS3 protease, the NS3 helicase, the NS5a protein, and/or the NS5b polymerase. In another embodiment, the therapy is the disruption of the interaction between the NS3 protease and NS4A cofactor. In another embodiment, the therapy is the prevention or alteration of the severing of one or more of the NS4A-NS4B, NS4B-NS5A and NS5A-NS5B junctions of the HCV. In another embodiment, the therapy is inhibition of the activity of a serine protease. In another embodiment, the therapy is reduction of the HCV RNA load of a subject.

In one aspect, the salts and crystalline forms of the invention exhibit HCV protease activity. In one embodiment, the salts and crystalline forms are HCV NS3-4A protease inhibitors.

In one aspect, the invention provides a method of inhibiting hepatitis C virus replication in a cell, comprising contacting said cell with a salt or crystalline form of the invention.

In another aspect, the invention provides a packaged HCV-associated disorder treatment, comprising a salt or crystalline form of the invention, packaged with instructions for using an effective amount of the salt or crystalline form to treat an HCV-associated disorder.

In certain embodiments, the HCV-associated disorder is selected from the group consisting of HCV infection, liver cirrhosis, chronic liver disease, hepatocellular carcinoma, cryoglobulinaemia, non-Hodgkin's lymphoma, liver fibrosis and a suppressed innate intracellular immune response.

In another embodiment, the invention provides a method of treating HCV infection, liver cirrhosis, chronic liver disease, hepatocellular carcinoma, cryoglobulinaemia, non-Hodgkin's lymphoma, liver fibrosis and/or a suppressed innate intracellular immune response in subject in need thereof comprising administering to the subject a pharmaceutically acceptable amount of a salt or crystalline form of the invention.

In one embodiment, the HCV to be treated is selected of any HCV genotype. In another embodiment, the HCV is selected from HCV genotype 1, 2 and/or 3.

HCV-associated states are often associated with the NS3 serine protease of HCV, which is responsible for several steps in the processing of the HCV polyprotein into smaller functional proteins. NS3 protease forms a heterodimeric complex with the NS4A protein, an essential cofactor that enhances enzymatic activity, and is believed to help anchor HCV to the endoplasmic reticulum. NS3 first autocatalyzes hydrolysis of the NS3-NS4A juncture, and then cleaves the HCV polyprotein intermolecularly at the NS4A-NS4B, NS4B-NS5A and NS5A-NS5B intersections. This process is associated with replication of HCV in a subject. Inhibiting or modulating the activity of one or more of the NS3, NS4A, NS4B, NS5A and NS5B proteins will inhibit or modulate replication of HCV in a subject, thereby preventing or treating the HCV-associated state. In a particular embodiment, the HCV-associated state is associated with the activity of the NS3 protease. In another particular embodiment, the HCV-associated state is associated with the activity of NS3-NS4A heterodimeric complex.

The invention also provides processes for the preparation of the crystalline forms described herein. Thus, in one aspect, the invention provides a process for the preparation of any of Forms A, B, C, D, E, F, G, H and I comprising the crystallisation of the Form from a solution of Compound X.

In the context of the present invention, references herein to “treatment” include references to curative, palliative and prophylactic treatment, unless there are specific indications to the contrary. The terms “therapy, “therapeutic” and “therapeutically” should be construed in the same way.

The salts and crystalline forms of the present invention may be administered alone or in combination with one or more other drugs. Generally, they will be administered as a formulation in association with one or more pharmaceutically acceptable excipients. The term “excipient” is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Pharmaceutical compositions suitable for the delivery of the salts and crystalline forms of the present invention and methods for their preparation will be readily apparent to those skilled in the art. Such compositions and methods for their preparation may be found, for example, in Remington's Pharmaceutical Sciences, 19th Edition (Mack Publishing Company, 1995).

For administration to human patients, the total daily dose of the salt or crystalline form is typically in the range 0.01 mg and 1000 mg, or between 0.1 mg and 250 mg, or between 1 mg and 50 mg depending, of course, on the mode of administration. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

The pharmaceutical compositions may be administered topically (e.g. to the skin or to the lung and/or airways) in the form, e.g., of creams, solutions, suspensions, heptafluoroalkane (HFA) aerosols and dry powder formulations; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

In an embodiment of the invention, the active ingredient is administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solid and liquid (including multiple phases or dispersed systems) such as tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Formulations suitable for oral administration may also be designed to deliver the salts and crystalline forms in an immediate release manner or in a rate-sustaining manner, wherein the release profile can be delayed, pulsed, controlled, sustained, or delayed and sustained or modified in such a manner which optimises the therapeutic efficacy of the active agent. Means to deliver compounds in a rate-sustaining manner are known in the art and include slow release polymers that can be formulated with the said compounds to control their release.

Examples of rate-sustaining polymers include degradable and non-degradable polymers that can be used to release the said compounds by diffusion or a combination of diffusion and polymer erosion. Examples of rate-sustaining polymers include hydroxypropyl methylcellulose, hydroxypropyl cellulose, methyl cellulose, ethyl cellulose, sodium carboxymethyl cellulose, polyvinyl alcohol, polyvinyl pyrrolidone, xanthum gum, polymethacrylates, polyethylene oxide and polyethylene glycol.

Liquid (including multiple phases and dispersed systems) formulations include emulsions, suspensions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The salts and crystalline forms of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L. Lachman (Marcel Dekker, New York, 1980).

### BRIEF DESCRIPTION OF THE FIGURES

The invention will now be illustrated by the following non-limiting examples. In the examples the following figures are presented:

FIG. 1: X-ray powder diffraction pattern of Form A, defined herein.

FIG. 2: X-ray powder diffraction pattern of Form B, defined herein

FIG. 3: X-ray powder diffraction pattern of Form C, defined herein.

FIG. 4: X-ray powder diffraction pattern of Form D, defined herein.

FIG. 5: X-ray powder diffraction pattern of Form E, defined herein.

FIG. 6: X-ray powder diffraction pattern of Form F, defined herein.

FIG. 7: X-ray powder diffraction pattern of Form G, defined herein.

FIG. 8: X-ray powder diffraction pattern of Form H, defined herein.

FIG. 9: X-ray powder diffraction pattern of Form I, defined herein.

FIG. 10: X-ray powder diffraction pattern of Form J, defined herein

FIG. 11: X-ray powder diffraction pattern of Form K, defined herein

FIG. 12: X-ray powder diffraction pattern of Form L, defined herein

FIG. 13: X-ray powder diffraction pattern of Form M, defined herein

FIG. 14: X-ray powder diffraction pattern of Form N, defined herein

### GENERAL EXPERIMENTAL DETAILS

### List of Abbreviations

Ac acetyl
ACN Acetonitrile
AcOEt/EtOAc Ethyl acetate
AcOH acetic acid
aq aqueous
Ar aryl
Bn benzyl
Bu butyl (nBu=n-butyl, tBu=tert-butyl)
CDI Carbonyldiimidazole
CH₃CN Acetonitrile
DBU 1,8-Diazabicyclo[5.4.0]-undec-7-ene
DCE 1,2-Dichloroethane
DCM Dichloromethane
DIPEA N-Ethyldiisopropylamine
DMAP Dimethylaminopyridine
DMF N,N′-Dimethylformamide
DMSO Dimethylsulfoxide
EI Electrospray ionisation
Et₂O Diethylether
Et₃N Triethylamine
Ether Diethylether
EtOH Ethanol
FC Flash Chromatography
h hour(s)
HATU O-(7-Azabenzotriazole-1-yl)-N,N,N′N′-tetramethyluronium hexafluorophosphate
HBTU O-(Benzotriazol-1-yl)-N,N,N′,N′-tetramethyluronium hexafluorophosphate
HCl Hydrochloric acid
HOBt 1-Hydroxybenzotriazole
HPLC High Performance Liquid Chromatography
H₂O Water
IPA Isopropyl alcohol
L liter(s)
LC-MS Liquid Chromatography Mass Spectrometry
Me methyl
MeI Iodomethane
MeOH Methanol
mg milligram
min minute(s)
mL milliliter
MS Mass Spectrometry
Pd/C palladium on charcoal
PG protecting group
Ph phenyl
Prep Preparative
Rf ratio of fronts
RP reverse phase
Rt Retention time
rt Room temperature
SiO₂ Silica gel
TBAF Tetrabutylammonium fluoride
TEA Triethylamine
TFA Trifluoroacetic acid
THF Tetrahydrofurane
TLC Thin Layer Chromatography

X-Ray Powder Diffraction (XRPD) patterns were collected using sample weights of approximately 2-10 mg, which was gently compressed on the XRPD zero background single obliquely cut silica sample holder. The sample was then loaded into a Bruker GADDS and analysed using the following experimental conditions:
Tube anode: (Cu)
Generator tension: 40 kV
Tube current: 40 mA
Start angle [2 θ]: 3
End angle [2 θ]: 4
Scan time 2 minutes

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

### Method B:

HPLC

Instrument: Agilent system

Column: Waters Symmetry C18, 3.5 microm., 2.1×50 mm, flow 0.6 mL/min

Solvent: CH₃CN (0.1% CF₃CO₂H), H₂O (0.1% CF₃CO₂H)

Gradient: 0-3.5 min: 20-95% CH₃CN, 3.5-5 min: 95% CH₃CN, 5.5-5.55 min 95% to 20% CH₃CN

### Method G:

LCMS

Instrument: Agilent system

Column: Halo C18, 2.7 microm., 2.1×30 mm, flow 1.1 mL/min

Solvent: CH₃CN (0.1% HCO₂H), H₂O (0.1% HCO₂H)

Gradient: 0-2 min: 5-95% CH₃CN, 2-2.6 min: 95% CH₃CN, 2.6-2.65 min 95% to 5% CH₃CN, 2.65-3 min 5% CH₃CN

### Method J:

MS

Instrument: Agilent system

Method: Flow injection

Detection: API-ES, positive/negative

### Compound X

The preparation of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide, as described in unpublished patent application no. PCT/IB2010/000784, is detailed below.

### Preparation of Reactant A

### Step 1a:

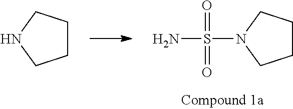 [SMILES: C1CCNC1 ; NS(=O)(=O)N1CCCC1]

A suspension of N-(tert-butoxycarbonyl)-N-[4-(dimethylazaniumylidene)-1,4-dihydropyridin-1-ylsulfonyl]azanide (3 g; 9.955 mmol) prepared according to the procedure from Winum et al (Organic Letters 2001, 3, 2241) in DCM (24 mL) was treated with pyrrolidine (0.864 mL; 10.453 mmol) and stirred at rt for 24 h. The reaction mixture was chromatographed by FC on silica gel (eluent: CH₂Cl₂/EtOAc 100:1) to give [N-(tert-butoxycarbonyl)]-pyrrolidine-1-sulfonic acid amide. TLC: Rf (DCM/EtOAc 100:1)=0.40. A solution of [N-(tert-butoxycarbonyl)]-pyrrolidine-1-sulfonic acid amide (57.09 g; 223 mmol) in DCM (450 mL) was treated with TFA (120 mL; 1.56 mol) and stirred at rt for 7 h. The reaction mixture was concentrated in vacuo and the residual oil was triturated with diisopropylether. The resulting powder was washed with diisopropylether and dried under high vacuum to provide Compound 1a. TLC: Rf (DCM/EtOAc 50:1)=0.10.

### Step 1b:

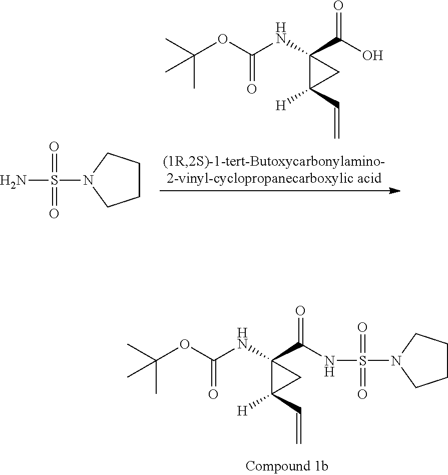 [SMILES: C=C[C@@H]1C[C@]1(NC(=O)OC(C)(C)C)C(=O)O ; NS(=O)(=O)N1CCCC1 ; C=C[C@@H]1C[C@]1(NC(=O)OC(C)(C)C)C(=O)NS(=O)(=O)N1CCCC1]

A solution of (1R,2S)-1-tert-butoxycarbonylamino-2-vinyl-cyclopropanecarboxylic acid prepared according to the procedure described in WO2000/09558 (8.24 g; 36.3 mmol) in THF (160 mL) was treated with CDI (9.09 g; 54.4 mmol) and heated to reflux for 1 h. The resulting reaction mixture was cooled to rt and treated with Compound 1a (7.62 g; 50.8 mmol) followed by DBU (8.28 g; 54.4 mmol). After 16 h at rt the reaction mixture was concentrated, the residue was taken up in DCM and washed with a saturated aq solution of KHSO₄ (3×). The aq phases were extracted with DCM, the organics were combined, dried over Na₂SO₄ and concentrated in vacuo. The residue was chromatographed on silica gel (eluent: hexane/EtOAc 4:1) to give Compound 1b. LCMS (method F) Rt=3.21 min; MS (method J): M/z=358 [M−1]

### Step 1c:

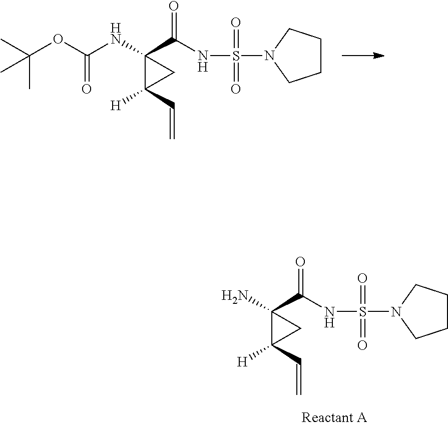 [SMILES: C=C[C@@H]1C[C@]1(NC(=O)OC(C)(C)C)C(=O)NS(=O)(=O)N1CCCC1 ; C=C[C@@H]1C[C@]1(N)C(=O)NS(=O)(=O)N1CCCC1]

Compound 1b (7.84 g; 21.81 mmol) was treated with 4N HCl in dioxane (84 mL) at rt. After 1.5 h the reaction mixture was concentrated under high vacuum to give Compound 1c as its hydrochloride salt. LCMS (method E) Rt=1.10 min; MS (method J): M/z=260 [M+1]

### Preparation of Intermediate II

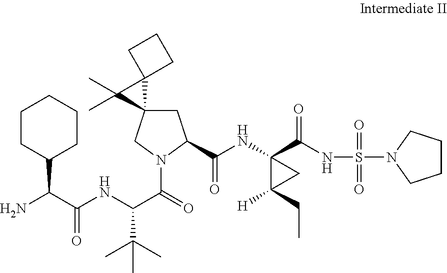 [SMILES: CC[C@@H]1C[C@]1(NC(=O)[C@@H]1C[C@@]2(CN1C(=O)[C@@H](NC(=O)[C@@H](N)C1CCCCC1)C(C)(C)C)C(C)(C)C21CCC1)C(=O)NS(=O)(=O)N1CCCC1]

### Step 2a:

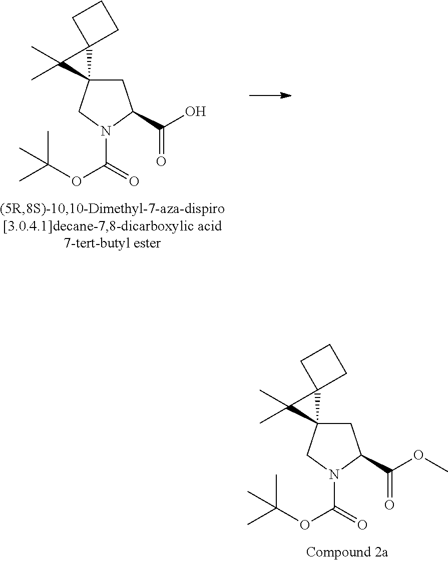 [SMILES: CC(C)(C)OC(=O)N1C[C@]2(C[C@H]1C(=O)O)C(C)(C)C21CCC1 ; COC(=O)[C@@H]1C[C@@]2(CN1C(=O)OC(C)(C)C)C(C)(C)C21CCC1]

(5R,8S)-10,10-Dimethyl-7-aza-dispiro[3.0.4.1]decane-7,8-dicarboxylic acid 7-tert-butyl ester (32.84 g; 106 mmol—prepared by the procedure described in WO2009/047264) in DMF (1 L) was treated with K₂CO₃ (22.00 g; 159 mmol) followed by methyliodide (9.93 mL; 159 mmol). The reaction mixture was stirred at rt for 18 h, concentrated in vacuo. The resulting residue was partitioned between water and EtOAc and extracted with EtOAc. The organics were combined, washed with brine, dried over Na₂SO₄ and concentrated. The residue was chromatographed on silica gel (eluent DCM/Ethylether 120:1) to give Compound 2a. TLC: Rf (DCM/Ethylether 120:1)=0.22; MS (method J): M/z=346 [M+Na]

### Step 2b:

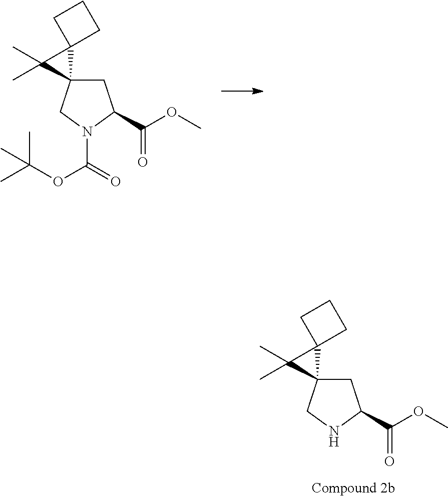

Compound 2b hydrochloride was obtained from Compound 2a (6.3 g; 19.48 mmol) by treatment with 4N HCl in dioxane (84 mL) at rt. After 1.5 h the reaction mixture was concentrated under high vacuum to give Compound 2b as its hydrochloride salt MS (method J): M/z=224 [M+1]

### Step 2c:

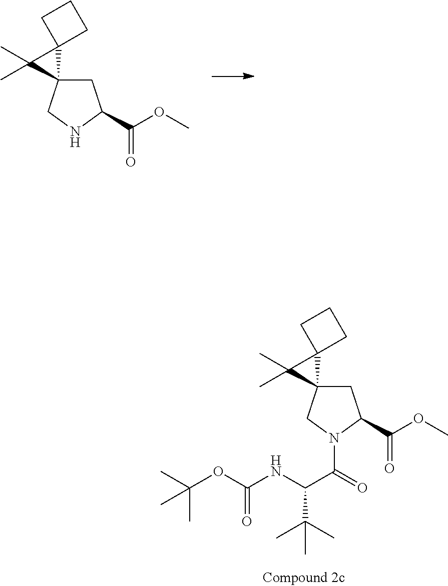

Compound 2c was obtained from a solution of Compound 2b hydrochloride (7.33 g; 27.93 mmol) and BOC-L-tert-leucine (0.248 g; 1.072 mmol) in DCM (15 mL) which was cooled to 0° C. and treated with DIPEA (0.46 mL; 2.68 mmol) and HATU (0.611 g; 1.608 mmol). The reaction mixture was stirred at rt for 20 h, concentrated in vacuo and the residue was purified by preparative HPLC (method K). After workup (Workup 2=fractions were treated with NaHCO₃ and concentrated; residue partitioned between water and EtOAc, extracted with EtOAc; organics combined, dried over Na₂SO₄ and concentrated) TLC: Rf (hexane/EtOAc 4:1)=0.37; MS (method J): M/z=437 [M+1]

### Step 2d:

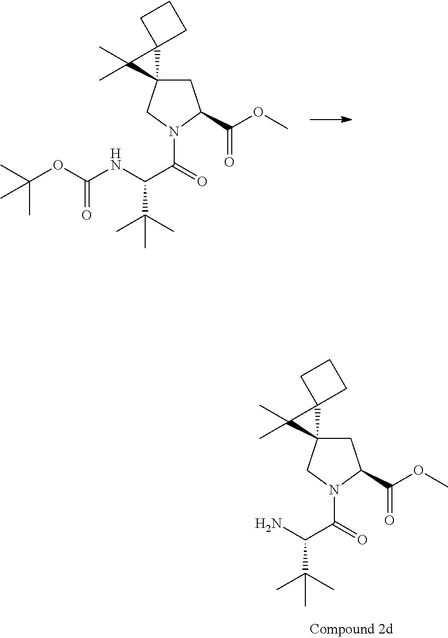

Compound 2d hydrochloride was obtained from Compound 2c (10.55 g; 24.16 mmol) by treatment with 4N HCl in dioxane (84 mL) at rt. After 1.5 h the reaction mixture was concentrated under high vacuum to give Compound 2d hydrochloride. TLC: Rf (DCM/MeOH 95:5)=0.39; MS (method J): M/z=337 [M+1]

### Step 2e:

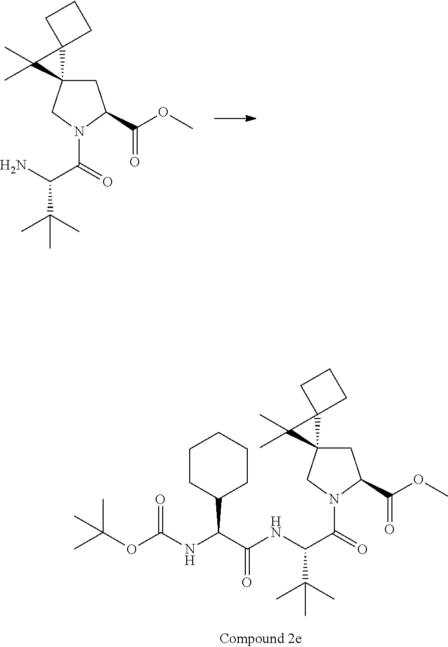

Compound 2e was obtained from Compound 2d (0.2 g; 0.456 mmol) and BOC-L-cyclohexylglycine (1.582 g; 6.15 mmol) in DCM (65 mL) which was cooled to 0° C. and treated with DIPEA (2.68 mL; 15.37 mmol) followed by HATU (3.51 g; 9.22 mmol). After 16 h at rt the reaction mixture was partitioned between DCM and 1N HCl, the organics were extracted with saturated aq NaHCO₃, dried over Na₂SO₄ and concentrated. Purification by preparative HPLC (method K) followed by workup (Workup 2) afforded Compound 2e. LC-MS (method G): Rt=2.21 min; M/z=598 [M+Na]

### Step 2f:

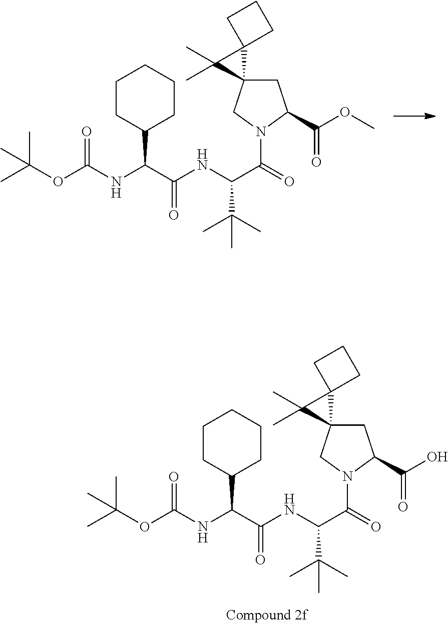

A mixture of Compound 2e (1.136 g; 1.973 mmol) and LiOH.H₂O (0.09 g; 2.17 mmol) in THF/MeOH/water (6 mL; 2:1:1) was stirred at rt 16 h. The reaction mixture was partitioned between water and EtOAc. The aq phase was acidified with 1N HCl and extracted with EtOAC. Organics were combined, dried over Na₂SO₄ and concentrated to a residue that was chromatographed on silica gel (DCM/MeOH 100% to 9:1) to afford Compound 2f. LC-MS (method G): Rt=1.99 min; M/z=562 [M+1]

### Step 2g:

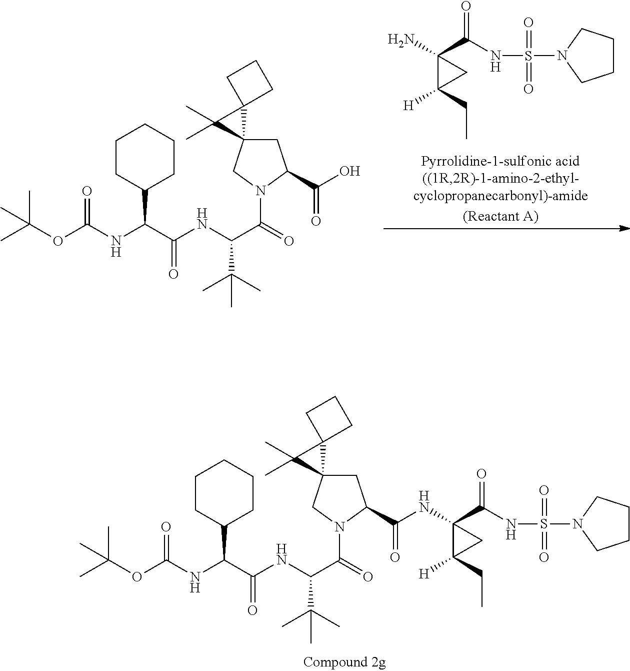 [SMILES: CC(C)(C)OC(=O)N[C@H](C(=O)N[C@H](C(=O)N1C[C@]2(C[C@H]1C(=O)O)C(C)(C)C21CCC1)C(C)(C)C)C1CCCCC1 ; CC[C@@H]1C[C@]1(N)C(=O)NS(=O)(=O)N1CCCC1 ; CC[C@@H]1C[C@]1(NC(=O)[C@@H]1C[C@@]2(CN1C(=O)[C@@H](NC(=O)[C@@H](NC(=O)OC(C)(C)C)C1CCCCC1)C(C)(C)C)C(C)(C)C21CCC1)C(=O)NS(=O)(=O)N1CCCC1]

A solution of Compound 2f (0.050 g; 0.089 mmol) and pyrrolidine-1-sulfonic acid ((1R,2R)-1-amino-2-ethyl-cyclopropanecarbonyl)-amide—Reactant A (0.030 g; 0.093 mmol—prepared as set out in the procedure herein) in DCM (2 mL) was cooled to 0° C. and treated with DIPEA (0.078 mL; 0.445 mmol) and HATU (0.102 g; 0.267 mmol). The reaction mixture was stirred at rt for 2 h, partitioned between DCM and 1N HCl. The organics were washed with a saturated aq NaHCO₃ solution, dried over Na₂SO₄ and concentrated in vacuo to a residue that was purified by preparative HPLC. After workup Compound 2g was obtained. LC-MS (method G): Rt=2.25 min; M/z=828 [M+Na]

### Step 2h:

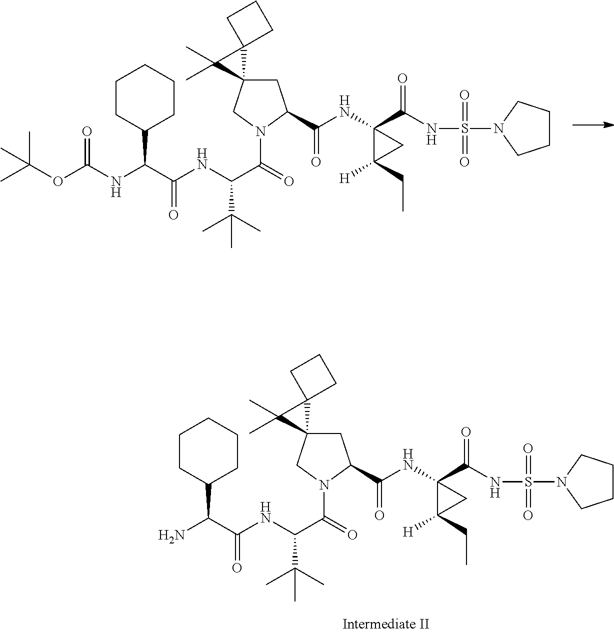 [SMILES: CC[C@@H]1C[C@]1(NC(=O)[C@@H]1C[C@@]2(CN1C(=O)[C@@H](NC(=O)[C@@H](NC(=O)OC(C)(C)C)C1CCCCC1)C(C)(C)C)C(C)(C)C21CCC1)C(=O)NS(=O)(=O)N1CCCC1 ; CC[C@@H]1C[C@]1(NC(=O)[C@@H]1C[C@@]2(CN1C(=O)[C@@H](NC(=O)[C@@H](N)C1CCCCC1)C(C)(C)C)C(C)(C)C21CCC1)C(=O)NS(=O)(=O)N1CCCC1]

Intermediate II hydrochloride was obtained from Compound 2g (0.02 g; 0.025 mmol) by treatment with 4N HCl in dioxane (84 mL) at rt. After 1.5 h the reaction mixture was concentrated under high vacuum to give Intermediate II hydrochloride. LC-MS (method G): Rt=1.59 min; M/z=706 [M+1]

### Synthesis of Compound X

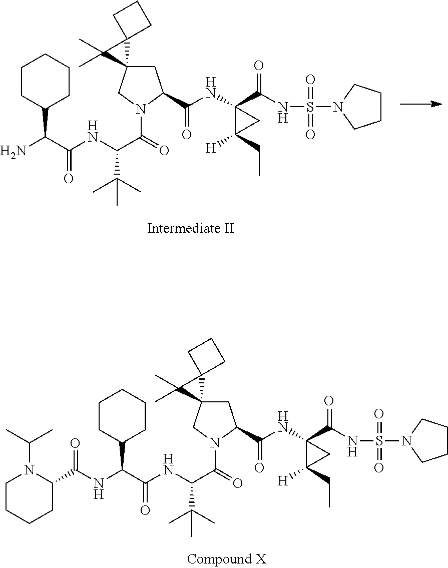 [SMILES: CC[C@@H]1C[C@]1(NC(=O)[C@@H]1C[C@@]2(CN1C(=O)[C@@H](NC(=O)[C@@H](N)C1CCCCC1)C(C)(C)C)C(C)(C)C21CCC1)C(=O)NS(=O)(=O)N1CCCC1 ; CC[C@@H]1C[C@]1(NC(=O)[C@@H]1C[C@@]2(CN1C(=O)[C@@H](NC(=O)[C@@H](NC(=O)[C@@H]1CCCCN1C(C)C)C1CCCCC1)C(C)(C)C)C(C)(C)C21CCC1)C(=O)NS(=O)(=O)N1CCCC1]

A suspension of (S)-1-isopropyl-piperidine-2-carboxylic acid (1.39 g; 8.11 mmol) in DMF (150 mL) was treated with HATU (3.86 g; 10.14 mmol) and DIPEA (3.54 mL; 20.29 mmol) and stirred at RT. The resulting solution was treated with Intermediate II hydrochloride ((0.28 g; 0.378 mmol) and stirred at RT under Argon for 1 h. The reaction mixture was taken up in EtOAc, washed with water. The aqueous phase was extracted with EtOAc. The organics were combined, washed with saturated aq NaHCO₃, dried over Na₂SO₄ and concentrated to a brown oil. Purification by FC on silica gel (eluent: cyclohexane to cyclohexane/aceton 3:2) afforded Compound X hydrochloride. HPLC (method B): Rt=3.70 min; MS (method J) M/z=858 [M+1] 1H-NMR (400 MHz, methanol-d4): δ (ppm)=8.4 (d, 1H), 4.75 (d, 1H), 4.3 (d, 1H), 4.2 (t, 1H), 3.95 (bs, 1H), 3.4-3.7 (m, 9H), 3.0 (m, 1H), 2.15 (m, 1H), 1.05-2.1 (m, 43H), 1.05 (s, 9H), 0.9 (s, 3H), 0.95 (s, 3H).

### Compound X HCl Salt (Form A)

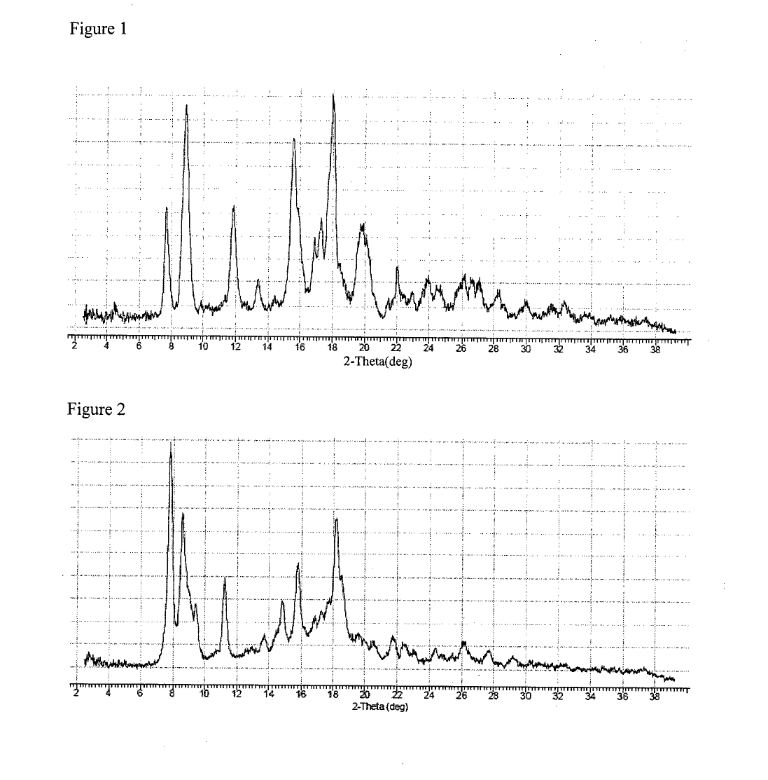

Dissolve free base in IPA and add 1 equivalent HCl. IPA is evaporated and the solids are equilibrated in acetonitrile at room temperature to give an di-hydrate form. The crystalline form (referred to herein as Form A) displayed the X-ray power diffraction peaks shown in Table A below. FIG. 1 shows the X-ray powder diffraction pattern of Form A.

TABLE A

| No. | Position | Intensity |
|---|---|---|
| 1 | 7.7 | 31 |
| 2 | 8.9 | 53 |
| 3 | 11.8 | 32 |
| 4 | 13.4 | 16 |
| 5 | 15.5 | 46 |
| 6 | 17.3 | 29 |
| 7 | 18.0 | 56 |
| 8 | 19.9 | 28 |
| 9 | 22.0 | 19 |
| 10 | 23.9 | 17 |
| 11 | 24.7 | 15 |
| 12 | 26.2 | 17 |
| 13 | 27.1 | 16 |
| 14 | 28.4 | 14 |

### Compound X HCl Salt (Form B)

Dissolve 600 mg of fee base in 3 ml ethyl acetate/acetonitrile (2:1) and add 700 μl 6N HCl. Solution was equilibrated for 1 hour then evaporated. The solids were resuspended in acetonitrile and slurried for 1 hour before isolation to give an anhydrous form. The crystalline form (referred to herein as Form B) displayed the X-ray power diffraction peaks shown in Table B below. FIG. 2 shows the X-ray powder diffraction pattern of Form B.

TABLE B

| No. | Position | Intensity |
|---|---|---|
| 1 | 7.8 | 87 |
| 2 | 8.6 | 62 |
| 3 | 9.5 | 30 |
| 4 | 11.3 | 40 |
| 5 | 13.7 | 20 |
| 6 | 14.8 | 32 |
| 7 | 15.8 | 45 |
| 8 | 18.2 | 61 |

### Compound X HCl Salt (Form C)

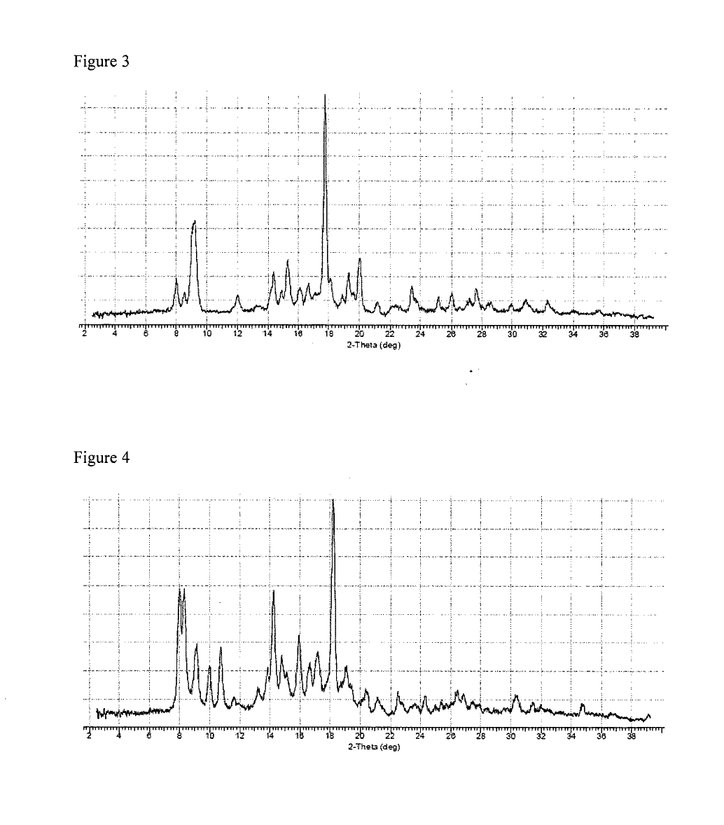

2.54 g of free base are dissolved in 3 ml acetone. 1.075 ml of 2.75 N HCl are added to the drug substance solution. This solution is equilibrated for 2 hours then solids were collected by filtration to give an anhydrous. The crystalline form (referred to herein as Form C) displayed the X-ray power diffraction peaks shown in Table C below. FIG. 3 shows the X-ray powder diffraction pattern of Form C.

TABLE C

| No. | Position | Intensity |
|---|---|---|
| 1 | 8.0 | 19 |
| 2 | 8.5 | 14 |
| 3 | 9.2 | 43 |
| 4 | 12.0 | 12 |
| 5 | 14.4 | 22 |
| 6 | 15.3 | 27 |
| 7 | 16.2 | 15 |
| 8 | 16.7 | 17 |
| 9 | 17.7 | 96 |
| 10 | 19.3 | 22 |
| 11 | 20.0 | 28 |
| 12 | 23.4 | 16 |
| 13 | 25.2 | 12 |
| 14 | 26.0 | 13 |

### Compound X HCl Salt (Form D)

The HCl salt (Form C) was equilibrated in ethyl acetate for 72 hours to give a di-hydrate form. The crystalline form (referred to herein as Form D) displayed the X-ray power diffraction peaks shown in Table D below. FIG. 4 shows the X-ray powder diffraction pattern of Form D.

TABLE D

| No. | Position | Intensity |
|---|---|---|
| 1 | 8.0 | 39 |
| 2 | 8.3 | 39 |
| 3 | 9.1 | 24 |
| 4 | 10.0 | 18 |
| 5 | 10.7 | 23 |
| 6 | 13.3 | 11 |
| 7 | 13.9 | 17 |
| 8 | 14.3 | 39 |
| 9 | 14.8 | 20 |
| 11 | 15.9 | 26 |
| 12 | 16.6 | 18 |
| 13 | 17.2 | 21 |
| 14 | 18.2 | 64 |
| 15 | 19.1 | 17 |
| 16 | 20.3 | 11 |
| 17 | 21.1 | 9 |
| 18 | 22.5 | 10 |
| 19 | 24.4 | 9 |

### Compound X HCl Salt (Form E)

The HCl salt was isolated after equilibration of Form B in water for 72 hours to give a tri-hydrate from. The crystalline form (referred to herein as Form E) displayed the X-ray power diffraction peaks shown in Table E below. FIG. 5 shows the X-ray powder diffraction pattern of Form E.

TABLE E

| No. | Position | Intensity |
|---|---|---|
| 1 | 7.5 | 32 |
| 2 | 7.9 | 12 |
| 3 | 8.8 | 52 |
| 4 | 9.2 | 47 |
| 5 | 10.4 | 8 |
| 6 | 12.6 | 10 |
| 8 | 14.4 | 9 |
| 9 | 15.6 | 27 |
| 10 | 16.7 | 13 |
| 11 | 17.8 | 38 |
| 12 | 18.2 | 19 |
| 13 | 19.5 | 16 |

### Compound X Hemi-HCl Salt (Form F)

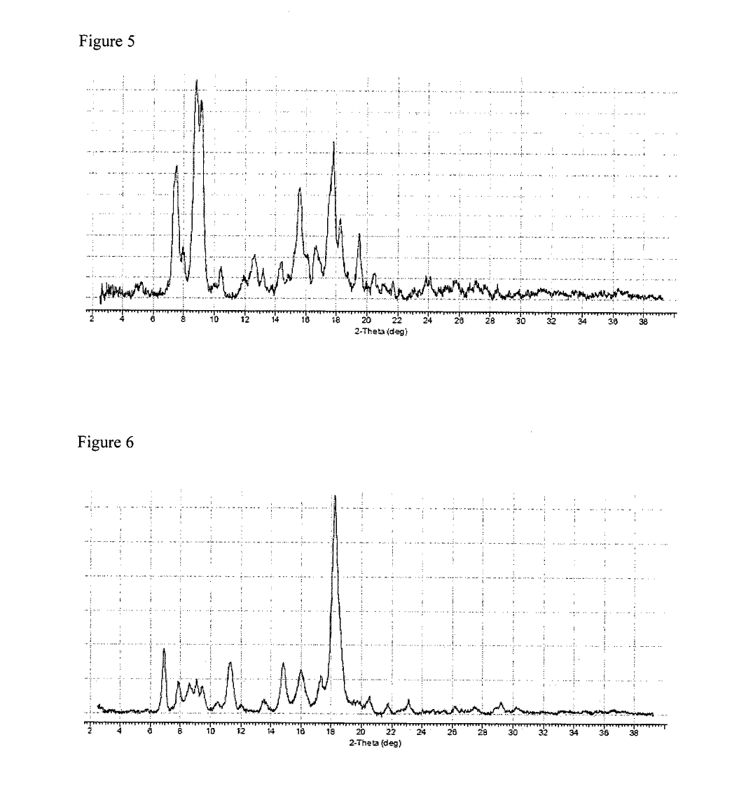

Dissolved 91 mg free base in 0.5 ml THF, added 53 μl 6N HCl. Added 2 ml acetonitrile and equilibrated the system for 4 hours before collecting solids to give a di-hydrate. The crystalline form (referred to herein as Form F) displayed the X-ray power diffraction peaks shown in Table F below. FIG. 6 shows the X-ray powder diffraction pattern of Form F.

TABLE F

| No. | Position | Intensity |
|---|---|---|
| 1 | 6.9 | 38 |
| 2 | 7.8 | 19 |
| 3 | 8.6 | 18 |
| 4 | 9.1 | 19 |
| 5 | 9.4 | 16 |
| 6 | 11.3 | 30 |
| 10 | 13.6 | 8 |
| 11 | 14.8 | 30 |
| 12 | 16.0 | 26 |
| 13 | 17.4 | 21 |
| 14 | 18.2 | 127 |
| 15 | 20.6 | 10 |

### Compound X MeSO₃H Salt (Form G)

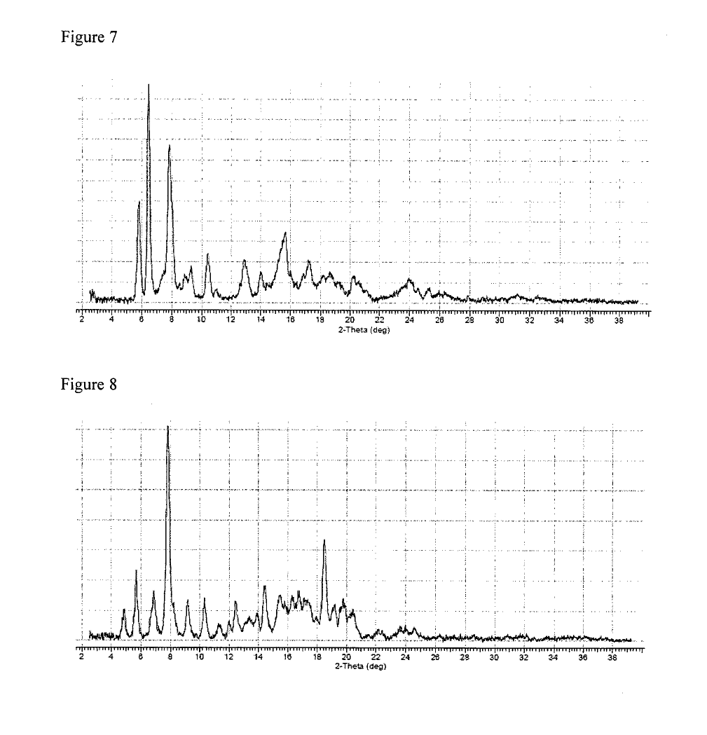

Dissolved 50 mg free base in methyl i-butyl ketone, added 1 equivalent methanesulfonic acid and equilibrated for 2 hours before collecting solids. The crystalline form (referred to herein as Form G) displayed the X-ray power diffraction peaks shown in Table G below. FIG. 7 shows the X-ray powder diffraction pattern of Form G.

TABLE G

| No. | Position | Intensity |
|---|---|---|
| 1 | 5.8 | 40 |
| 2 | 6.5 | 86 |
| 3 | 7.8 | 62 |
| 4 | 8.9 | 11 |
| 5 | 9.3 | 14 |
| 6 | 10.4 | 20 |
| 10 | 12.9 | 17 |
| 11 | 14.0 | 12 |
| 12 | 15.7 | 28 |
| 13 | 17.2 | 17 |
| 14 | 18.6 | 12 |
| 15 | 20.3 | 11 |

### Compound X Free Base (Form H)

Compound X free base is precipitated from isopropyl acetate (clear solution) during solvent-exchange with CH₃CN (suspension) and stirred at 20° C. for 24 h. The final drug substance was isolated from suspension of CH₃CN and washed by CH₃CN to give an anhydrous form. The crystalline form (referred to herein as Form H) displayed the X-ray power diffraction peaks shown in Table H below. FIG. 8 shows the X-ray powder diffraction pattern of Form H.

TABLE H

| No. | Position | Intensity |
|---|---|---|
| 1 | 4.9 | 11 |
| 2 | 5.7 | 21 |
| 3 | 6.9 | 16 |
| 4 | 7.8 | 60 |
| 5 | 9.2 | 14 |
| 6 | 10.3 | 15 |
| 7 | 12.0 | 9 |
| 8 | 12.4 | 15 |
| 9 | 13.3 | 10 |
| 10 | 13.9 | 12 |
| 11 | 14.4 | 19 |
| 12 | 15.5 | 17 |
| 13 | 16.3 | 17 |
| 14 | 16.7 | 18 |
| 15 | 18.5 | 32 |
| 16 | 19.2 | 15 |
| 17 | 19.8 | 16 |
| 18 | 20.3 | 12 |

### Compound X (Form I)

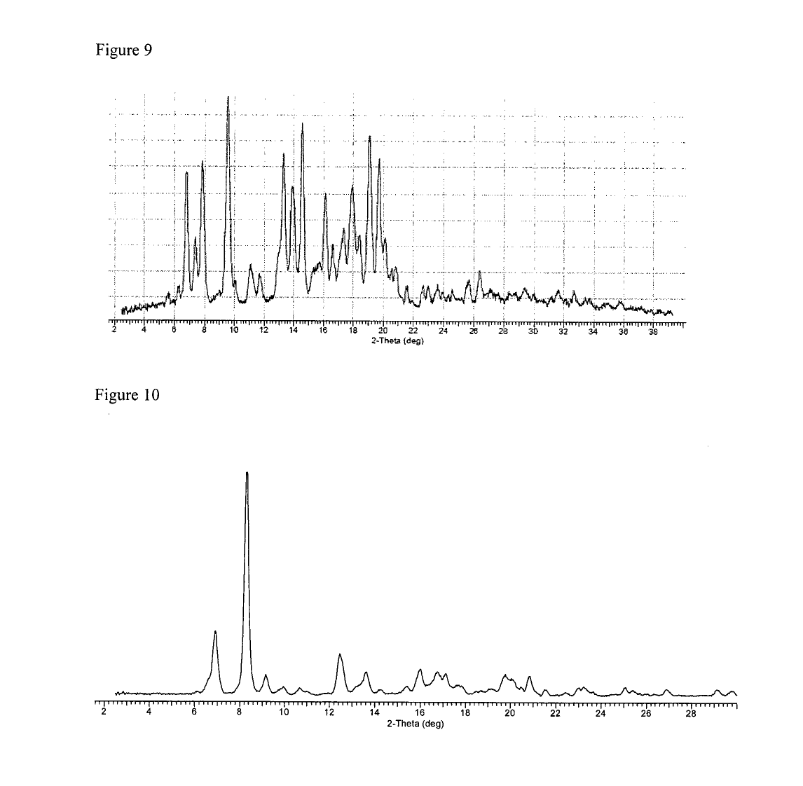

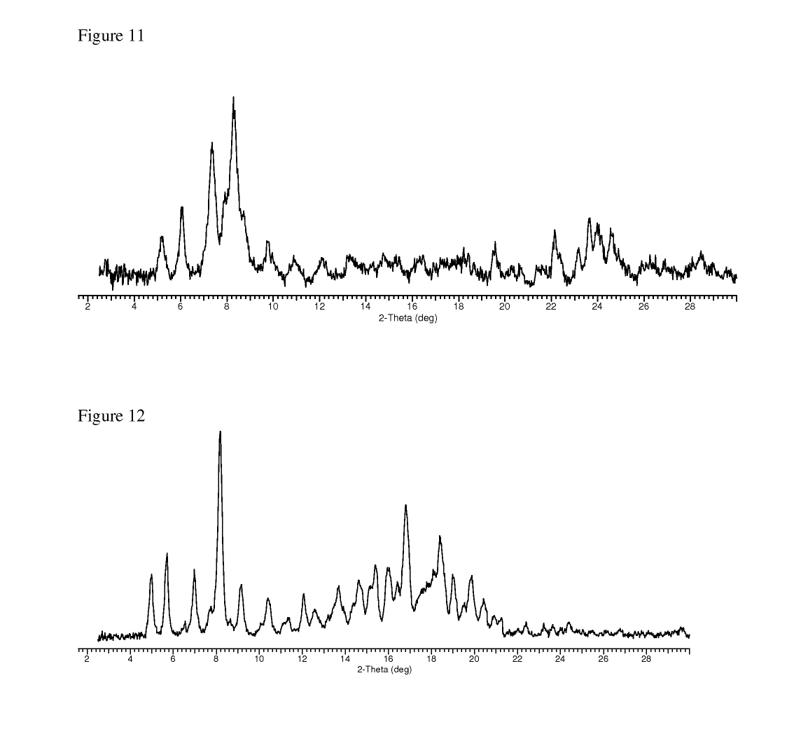

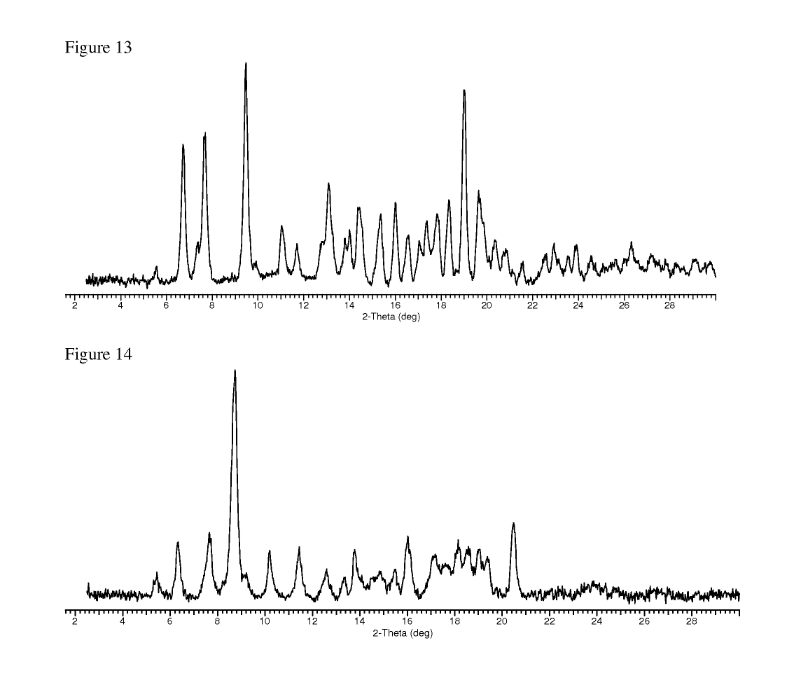

Form I is prepared by dissolving the drug substance in ethanol and precipitating with water to a final ratio of 1:1 to give a mono-hydrate form. The crystalline form (referred to herein as Form I) displayed the X-ray power diffraction peaks shown in Table I below. FIG. 9 shows the X-ray powder diffraction pattern of Form I.

TABLE I

| No. | Position | Intensity |
|---|---|---|
| 1 | 5.5 | 9 |
| 2 | 6.3 | 9 |
| 3 | 6.8 | 76 |
| 4 | 7.3 | 21 |
| 5 | 7.8 | 69 |
| 6 | 9.6 | 100 |
| 7 | 11.0 | 24 |
| 8 | 11.7 | 20 |
| 9 | 13.3 | 64 |
| 10 | 13.8 | 36 |
| 11 | 14.6 | 79 |
| 12 | 15.2 | 15 |
| 13 | 15.7 | 18 |
| 14 | 16.1 | 45 |
| 15 | 16.5 | 23 |
| 16 | 17.3 | 31 |
| 17 | 17.8 | 61 |
| 18 | 18.4 | 43 |
| 19 | 19.0 | 90 |
| 20 | 19.6 | 49 |
| 21 | 20.0 | 42 |
| 22 | 20.5 | 15 |
| 23 | 20.7 | 15 |
| 24 | 21.5 | 9 |
| 25 | 22.5 | 8 |
| 26 | 23.0 | 9 |
| 27 | 23.5 | 10 |
| 28 | 26.4 | 12 |

### Compound X (Form J))

Form J obtained from methanol by equilibrating form H at 50° C. Solids are collected by filtration and dried at 50 C.

TABLE J

| No. | Position | Relative I |
|---|---|---|
| 1 | 6.9 | 28 |
| 2 | 8.3 | 100 |
| 3 | 9.2 | 9 |
| 4 | 10.0 | 4 |
| 5 | 10.7 | 3 |
| 6 | 12.5 | 18 |
| 7 | 13.6 | 10 |
| 8 | 14.3 | 3 |
| 9 | 15.4 | 4 |
| 10 | 16.0 | 11 |
| 11 | 16.8 | 10 |
| 12 | 17.1 | 10 |
| 13 | 17.7 | 5 |
| 14 | 19.1 | 3 |
| 15 | 19.8 | 9 |
| 16 | 20.9 | 9 |
| 17 | 21.6 | 3 |
| 18 | 23.0 | 4 |
| 19 | 23.2 | 4 |
| 20 | 25.0 | 4 |
| 21 | 26.9 | 3 |
| 22 | 29.1 | 3 |

### Compound X (Form K)

Form K was crystallized from ethanol (100%) after the addition of acetonitrile. Solids were collected by centrifugation and dried under nitrogen flow.

TABLE K

| No. | Position | Relative I |
|---|---|---|
| 1 | 5.2 | 26 |
| 2 | 6.1 | 42 |
| 3 | 7.4 | 76 |
| 4 | 8.3 | 100 |
| 5 | 9.7 | 24 |
| 6 | 10.9 | 16 |
| 7 | 12.1 | 14 |
| 8 | 13.2 | 14 |
| 9 | 14.7 | 17 |
| 10 | 16.3 | 16 |
| 11 | 18.2 | 20 |
| 12 | 19.6 | 24 |
| 13 | 22.1 | 30 |
| 14 | 23.2 | 20 |
| 15 | 23.7 | 36 |
| 16 | 24.1 | 33 |
| 17 | 24.6 | 31 |
| 18 | 28.5 | 19 |

### Compound X (Form L)

Form L is the hydrated form of form H produced from acetonitrile and solids dried at 50 C. Exposing the solid to elevated humidity generates form L.

TABLE L

| No. | Position | Relative I |
|---|---|---|
| 1 | 5.0 | 29 |
| 2 | 5.7 | 46 |
| 3 | 7.0 | 31 |
| 4 | 7.8 | 25 |
| 5 | 8.2 | 100 |
| 6 | 8.7 | 12 |
| 7 | 9.2 | 27 |
| 8 | 10.4 | 20 |
| 9 | 11.4 | 13 |
| 10 | 12.1 | 21 |
| 11 | 12.7 | 16 |
| 12 | 13.7 | 24 |
| 13 | 14.7 | 24 |
| 14 | 15.4 | 30 |
| 15 | 16.0 | 30 |
| 16 | 16.4 | 26 |
| 17 | 16.9 | 57 |
| 18 | 17.6 | 23 |
| 19 | 17.9 | 25 |
| 20 | 18.1 | 29 |
| 21 | 18.4 | 42 |
| 22 | 18.5 | 43 |
| 23 | 19.1 | 24 |
| 24 | 19.9 | 28 |
| 25 | 20.4 | 18 |
| 26 | 20.9 | 13 |
| 27 | 22.1 | 10 |
| 28 | 22.4 | 11 |
| 29 | 23.3 | 11 |
| 30 | 24.4 | 12 |

### Compound X (Form M)

Form M is isolated from ethanol by precipitation with water. Solids are isolated by filtration and dried at 50 C. Exposing the solid to elevated humidity generates form M.

TABLE M

| No. | Position | Relative I |
|---|---|---|
| 1 | 5.6 | 9 |
| 2 | 6.7 | 63 |
| 3 | 7.3 | 18 |
| 4 | 7.6 | 67 |
| 5 | 7.7 | 69 |
| 6 | 9.5 | 100 |
| 7 | 11.0 | 27 |
| 8 | 11.7 | 19 |
| 9 | 13.1 | 46 |
| 10 | 13.8 | 21 |
| 11 | 14.0 | 25 |
| 12 | 14.4 | 35 |
| 13 | 15.4 | 32 |
| 14 | 16.0 | 37 |
| 15 | 16.6 | 23 |
| 16 | 17.0 | 20 |
| 17 | 17.4 | 29 |
| 18 | 17.8 | 32 |
| 19 | 18.3 | 38 |
| 20 | 19.0 | 88 |
| 21 | 19.6 | 43 |
| 22 | 20.4 | 21 |
| 23 | 20.8 | 17 |
| 24 | 21.6 | 11 |
| 25 | 22.5 | 14 |
| 26 | 22.9 | 19 |
| 27 | 23.5 | 13 |
| 28 | 23.9 | 18 |
| 29 | 24.6 | 13 |
| 30 | 26.3 | 20 |
| 31 | 27.2 | 14 |
| 32 | 29.1 | 12 |

### Compound X (Form N)

From N was crystallized from ethanol (100%) after the addition of acetonitrile. Solids were collected by centrifugation and dried under nitrogen flow.

TABLE N

| No. | Position | Relative I |
|---|---|---|
| 1 | 5.4 | 12 |
| 2 | 6.3 | 25 |
| 3 | 7.7 | 29 |
| 4 | 8.7 | 100 |
| 5 | 9.2 | 12 |
| 6 | 10.2 | 22 |
| 7 | 11.4 | 23 |
| 8 | 12.6 | 14 |
| 9 | 13.3 | 10 |
| 10 | 13.8 | 22 |
| 11 | 14.8 | 13 |
| 12 | 15.5 | 14 |
| 13 | 16.0 | 28 |
| 14 | 17.2 | 20 |
| 15 | 17.7 | 15 |
| 16 | 18.1 | 26 |
| 17 | 18.6 | 22 |
| 18 | 19.0 | 22 |
| 19 | 19.4 | 19 |
| 20 | 20.5 | 34 |

### Biological Activity

### HCV NS3-4A Protease Assay

The inhibitory activity of Compound X against HCV NS3-4A serine protease is determined in a homogenous assay using the full-length NS3-4A protein (genotype 1a, strain HCV-1) and a commercially available internally-quenched fluorogenic peptide substrate as described by Taliani, M., et al. 1996 Anal. Biochem. 240:60-67, which is incorporated by reference in its entirety.

### Luciferase-Based HCV Replicon Assay

The antiviral activity and cytotoxicity of Compound X is determined using a subgenomic genotype 1b HCV replicon cell line (Huh-Luc/neo-ET) containing a luciferase reporter gene, the expression of which is under the control of HCV RNA replication and translation. Briefly, 5,000 replicon cells are seeded in each well of 96-well tissue culture plates and are allowed to attach in complete culture media without G418 overnight. On the next day, the culture media are replaced with media containing serially diluted Compound X in the presence of 10% FBS and 0.5% DMSO. After a 48-h treatment with the compound, the remaining luciferase activities in the cells are determined using BriteLite reagent (Perkin Elmer, Wellesley, Mass.) with a LMaxII plate reader (Molecular Probe, Invitrogen). Each data point represents the average of four replicates in cell culture. IC₅₀ is the concentration of the compound at which the luciferase activity in the replicon cells is reduced by 50%. The cytotoxicity of the compound is evaluated using an MTS-based cell viability assay.

Compound X has been tested in the protease assay above. The IC₅₀ value is provided below. Compound X has also been tested in the replicon assay above and exhibits an IC₅₀ of less than about 100 nM or less.

| Protease | | | | | |
|---|---|---|---|---|---|
| assay | LC- | Retention | MS- | Mass | |
| IC50 (μM) | Method | time | method | observed | Ion |
| 0.0045 | B | 3.7 | J | 858.5 | M + H |

We claim:

1. (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide hydrochloride salt, or a pharmaceutically acceptable derivative thereof.

2. (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide hemi-hydrochloride salt, or a pharmaceutically acceptable derivative thereof.

3. (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide methanesulphonic acid salt, or a pharmaceutically acceptable derivative thereof.

4. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide hydrochloride salt (Form A) which exhibits at least the following characteristic X-ray powder diffraction peaks (expressed in degrees 2θ): 7.7, 8.9, 11.8, 15.5 and 18.0.

5. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide hydrochloride salt (Form B) which exhibits at least the following characteristic X-ray powder diffraction peaks (expressed in degrees 2θ): 7.8, 8.6, 11.3, 15.8 and 18.2.

6. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide hydrochloride salt (Form C) which exhibits at least the following characteristic X-ray powder diffraction peaks (expressed in degrees 2θ): 9.2, 14.4, 15.3, 17.7 and 20.0.

7. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide hydrochloride salt (Form D) which exhibits at least the following characteristic X-ray powder diffraction peaks (expressed in degrees 2θ): 8.0, 8.3, 14.3, 15.9 and 18.2.

8. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide hydrochloride salt (Form E) which exhibits at least the following characteristic X-ray powder diffraction peaks (expressed in degrees 2θ): 7.5, 8.8, 9.2, 15.6 and 17.8.

9. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide hemi-hydrochloride salt (Form F) which exhibits at least the following characteristic X-ray powder diffraction peaks (expressed in degrees 2θ): 6.9, 11.3, 14.8, 16.0 and 18.2.

10. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide methanesulfonic acid salt (Form G) which exhibits at least the following characteristic X-ray powder diffraction peaks (expressed in degrees 2θ): 5.8, 6.5, 7.8, 10.4 and 15.7.

11. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide (Form H) which exhibits at least the following characteristic X-ray powder diffraction peaks (expressed in degrees 2θ): 5.7, 6.9, 7.8, 14.4 and 18.5

12. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide (Form I) which exhibits at least the following characteristic X-ray powder diffraction peaks (expressed in degrees 2θ): 9.5, 13.3, 14.5, 19.0 and 19.7.

13. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide (Form J) which exhibits at least the following characteristic X-ray powder diffraction peaks (expressed in degrees 2θ): 6.9, 8.3, 12.5, 13.6, 16.0, 16.8, and 17.1.

14. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide (Form K) which exhibits at least the following characteristic X-ray powder diffraction peaks (expressed in degrees 2θ): 6.1, 7.4, 8.3, 22.1, 23.7, 24.1, and 24.6.

15. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide (Form L) which exhibits at least the following characteristic X-ray powder diffraction peaks (expressed in degrees 2θ): 5.7, 8.2, 16.9, 18.4 and 18.5.

16. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide (Form M) which exhibits at least the following characteristic X-ray powder diffraction peaks (expressed in degrees 2θ): 6.7, 7.6, 7.7, 9.5, and 19.0.

17. A crystalline form of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide (Form N) which exhibits at least the following characteristic X-ray powder diffraction peaks (expressed in degrees 2θ): 6.3, 7.7, 8.7, 16.0, 18.1, and 20.5.

18. A pharmaceutical composition, comprising:
a (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide crystalline form to claim 11, in association with a pharmaceutically acceptable adjuvant, diluent or carrier.

19-23. (canceled)

24. A method of treatment, comprising:
administering to a subject in need thereof a therapeutically effective amount of a (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide crystalline form to claim 11.

25. The method of claim 19, for treatment of a HCV-associated disorder.

26. The method of claim 19, wherein the HCV-associated disorder is selected from the group consisting of HCV infection, liver cirrhosis, chronic liver disease, hepatocellular carcinoma, cryoglobulinaemia, non-Hodgkin's lymphoma, liver fibrosis and a suppressed innate intracellular immune response.

27. A process for the preparation of crystalline Forms A, B, C, D, E, F, G, H, I, J, K, L, M or N of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide comprising
the crystallisation of the Form from a solution of (5R,8S)-7-[(2S)-2-{[(2S)-2-cyclohexyl-2-({[(2S)-1-isopropylpiperidin-2-yl]carbonyl}amino)acetyl]amino}-3,3-dimethylbutanoyl]-N-{(1R,2R)-2-ethyl-1-[(pyrrolidin-1-ylsulfonyl)carbamoyl]cyclopropyl}-10,10-dimethyl-7-azadispiro[3.0.4.1]decane-8-carboxamide.
